(19) United States
(12) Patent Application Publication
(10) Pub. No.: US 2025/0381390 A1
(43) Pub. Date: Dec. 18, 2025

(54) EXTRACTION DEVICES AND METHODS OF EXTRACTING IMPLANTED MEDICAL DEVICES

(71) Applicant: Merit Medical Systems, Inc., South Jordan, UT (US)

(72) Inventor: Barry E. Norlander, Freeport, PA (US)

(21) Appl. No.: 19/236,186

(22) Filed: Jun. 12, 2025

Related U.S. Application Data
(60) Provisional application No. 63/659,523, filed on Jun. 13, 2024.

(51) Int. Cl. A61N 1/05

(52) U.S. Cl. CPC A61N 1/057; A61N 2001/0578

(57) ABSTRACT

Extraction devices and methods of extracting an implanted medical device from a target location within a body lumen of an animal are provided. An extraction device comprises an outer sheath, an inner member disposed within, and axially movable relative to, the outer sheath, and a distal support member attached to the outer sheath and the inner member, the inner member comprising an engagement portion attached to the distal support member and movable between a first configuration having a first axial length and a first inner diameter and a second configuration having a second axial length that is greater than the first axial length and a second inner diameter that is less than the first inner diameter. A method of extracting an implanted medical device comprises inserting the distal end of an extraction device according to an embodiment into a body vessel of an animal; advancing the distal end of the extraction device to the target location and over the implanted medical device; engaging the implanted medical device with the engagement portion of the extraction device by retracting the inner member of the extraction device relative to the outer sheath of the extraction device, such as by application of a pulling force on the inner member of the extraction device while maintaining a position of the outer sheath of the extraction device; extracting the implanted medical device from tissue within which the medical device is implanted by retracting the extraction device within the body vessel; and withdrawing the extraction device and the implanted medical device from the body vessel.

### RELATED APPLICATIONS

The present application claims priority to U.S. Provisional Application No. 63/659,523, filed on Jun. 13, 2024 and titled, “Extraction Devices and Methods of Extracting Implanted Medical Devices,” which is hereby incorporated by reference in its entirety.

### TECHNICAL FIELD

The disclosure relates generally to the field of medical devices. More particularly, the disclosure relates to extraction devices useful for extracting implanted medical devices from a target location within a body lumen.

### BRIEF DESCRIPTION OF THE SEVERAL VIEWS OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a perspective view of an example extraction device, according to embodiments described herein.

FIG. 2 illustrates a lateral view of a distal end portion of the extraction device of FIG. 1, according to embodiments described herein. The engaging portion of the inner member is shown in a first configuration.

FIG. 3 illustrates a lateral view of the distal end portion of the extraction device of FIG. 1, according to embodiments described herein. The engaging portion of the inner member is shown in a second configuration.

FIG. 4 illustrates a sectional view of the extraction device of FIG. 2, taken along line 4-4, according to embodiments described herein.

FIG. 5A illustrates a sectional view of the extraction device of FIG. 2, taken along line 5-5, according to embodiments described herein.

FIG. 5B illustrates a sectional view of an exemplary embodiment of an extraction device, according to embodiments described herein.

FIG. 6 illustrates a sectional view of the extraction device of FIG. 2, taken along line 6-6, according to embodiments described herein.

FIG. 7 illustrates a sectional view of the extraction device of FIG. 3, taken along line 7-7, according to embodiments described herein.

FIG. 8 illustrates a lateral view of a distal end of the extraction device of FIG. 1, according to embodiments described herein. The engaging portion of the inner member is shown in the first configuration.

FIG. 9 illustrates a perspective view of an exemplary embodiment of an extraction device, according to embodiments described herein.

FIG. 10 illustrates a lateral view of a distal end portion of the example extraction device of FIG. 9 with the engaging portion in a first configuration, according to embodiments described herein.

FIG. 11 illustrates a lateral view of a distal end portion of the example extraction device of FIG. 9 with the engaging portion in a second configuration, according to embodiments described herein.

FIG. 12 illustrates an example method for extracting an implanted medical device, according to embodiments described herein.

FIG. 13 illustrates a schematic illustration of an extraction device disposed within a human heart and in a first position relative to implanted leads, according to embodiments described herein.

FIG. 14 illustrates a schematic illustration of an extraction device disposed within a human heart and in a second position relative to implanted leads, according to embodiments described herein.

FIG. 15 illustrates a schematic illustration of an extraction device disposed within a human heart as the pacing leads are being extracted relative to the heart, according to embodiments described herein.

FIG. 16 illustrates an example method for extracting an implanted medical device, according to embodiments described herein.

### DETAILED DESCRIPTION

Recent developments in cardiac electrophysiology and pacing modalities have resulted in medical devices, such as cardiac pacing leads, being implanted in structurally complex and precise locations within the vasculature and heart tissue. Within specific modalities, cardiac pacing leads-typically implanted into myocardial tissue—can be precisely placed so as to enhance activation patterns and improve patient outcomes. However, once placed, pacing leads can suffer from a host of issues that are resolved via extraction of the leads, or extraction of the implanted end of a lead from myocardial tissue. For instance, buildup of fibrotic tissue, device or lead malfunction, infection, and so on can at times be dealt with via extraction of the lead.

Specific placement and/or orientations of leads within heart anatomy can present challenges during extraction of device leads when necessary. For example, a conduction system pacing (CSP) therapy modality has been shown to improve clinical outcomes in patients with heart failure (HF) and other dyssynchronization focused arrhythmias. However, use of a CSP therapy modality requires implanting a cardiac pacing lead on or near the Bundle of His, the right bundle branch, the left bundle branch, or another post-atrioventricular (AV) node conduction pathway position.

In some cases, the lead target location is located at the upper area of the patient's ventricular septal region or at the lower area of the patient's atrial septal region. Each of these lead target locations for CSP pacing can create challenging structural stresses to the cardiac pacing lead. Furthermore, target locations for CSP pacing can position the pacing lead near chordae of the tricuspid valve, papillary muscles, and other fragile structures of the heart. Additionally, a CSP lead path may introduce bending and flexure between the distal electrodes of bipolar leads. In some cases, transvenous lead extraction (TLE) of leads implanted on a CSP lead path can be more challenging than extraction of leads implanted on other lead paths for a variety of reasons. For example, post failure, the distal tip and portion of conductor wire, insulation and other parts of a pacing lead implanted on a CSP lead path may stay affixed to the heart/septal wall in a post-AV node location and become a nidus for infection, arrythmias or embolism. Attempting to remove an affixed lead lip fragment can thus be challenging, since it must be grasped or snared secondarily to allow for a percutaneous extraction to be completed.

Aspects and implementations of the present disclosure address these and other challenges by providing extraction devices useful for extracting cardiac pacing leads implanted within complex cardiovascular anatomy. Furthermore, the present disclosure relates to methods of use, including extracting an implanted medical device from a target location within a body lumen of an animal.

In some embodiments, an extraction device can include an outer sheath, an inner member disposed within and axially movable relative to the outer sheath, and a distal support member attached to the outer sheath and the inner member. The inner member can include an engagement portion attached to the distal support member. The engagement portion can be movable between a first configuration having a first axial length and a first inner diameter, and a second configuration having a second axial length (that is greater than the first axial length) and a second inner diameter (that is less than the first inner diameter).

In alternate embodiments, an extraction device can include an outer sheath, an inner member disposed within and axially movable relative to the outer sheath, and a distal support member attached to the outer sheath and the inner member. The inner member can include an engagement portion including a braid of wires having a round cross-sectional shape. A distal end of the engagement portion can be embedded in the distal support member. The engagement portion can be movable between a first configuration having a first axial length and a first inner diameter and a second configuration having a second axial length (that is greater than the first axial length) and a second inner diameter (that is less than the first inner diameter). The inner member can include a first radiolucent marker disposed proximal to the engagement portion, and the distal support member can include a second radiolucent marker.

In alternate embodiments, an extraction device includes an outer sheath, an inner member disposed within and axially movable relative to the outer sheath, and a distal support member attached to the outer sheath (and the inner member) and comprising a radiolucent material. The inner member includes an engagement portion including a braid of wires of a shape memory material and having a round cross-sectional shape. A distal end of the engagement portion can be embedded in the distal support member, the engagement portion movable between a first configuration having a first axial length and a first inner diameter and a second configuration having a second axial length (that is greater than the first axial length) and a second inner diameter (that is less than the first inner diameter). The inner member includes a plurality of radiolucent markers disposed proximal to and adjacent the engagement portion.

In some embodiments, an example method of extracting an implanted medical device includes inserting the distal end of an extraction device according to an embodiment into a body vessel of an animal; advancing the distal end of the extraction device to the target location and over the implanted medical device; engaging the implanted medical device with the engagement portion of the extraction device by retracting the inner member of the extraction device relative to the outer sheath of the extraction device (e.g., such as by application of a pulling force on the inner member of the extraction device while maintaining a position of the outer sheath of the extraction device); extracting the implanted medical device from tissue within which the medical device is implanted by retracting the extraction device within the body vessel; and withdrawing the extraction device and the implanted medical device from the body vessel.

In some embodiments, an example method of extracting an implanted medical device includes connecting an accessory device to the implanted medical device; inserting the distal end of an extraction device according to an embodiment into a body vessel of an animal; advancing the extraction device over the accessory device; advancing the extraction device over the implanted medical device; engaging the implanted medical device with the engagement portion of the extraction device by retracting the inner member of the extraction device relative to the outer sheath of the extraction device (e.g., such as by application of a pulling force on the inner member of the extraction device while maintaining a position of the outer sheath of the extraction device); extracting the implanted medical device from tissue within which the medical device is implanted by retracting the extraction device within the body vessel; and withdrawing the extraction device, accessory device, and the implanted medical device from the body vessel.

Example methods using extraction devices according to embodiments described herein are particularly well suited for extraction of cardiac pacing leads implanted in the tissue of the heart under a CSP therapy modality, which places an implanted cardiac pacing lead in a post-AV node location within the heart. In some cases, an example method of extracting a cardiac pacing lead implanted in a post-AV node location of the heart includes connecting an accessory device to the implanted lead; advancing an extraction device according to an embodiment over the accessory device; advancing the distal end of the extraction device through the body vessel and into the right atrium of the heart of the animal; advancing the distal end of the extraction device through the tricuspid valve and distally toward the distal tip of the implanted cardiac pacing lead; advancing the distal end of the extraction device over the implanted cardiac pacing lead; engaging the implanted cardiac pacing lead with the engagement portion of the extraction device by retracting the inner member of the extraction device relative to the outer sheath of the extraction device (e.g., such as by application of a pulling force on the inner member of the extraction device while maintaining a position of the outer sheath of the extraction device); extracting the implanted cardiac pacing lead from an implantation location in tissue defining the right ventricle of the heart of the animal by retracting the extraction device within the body vessel; retracting the extraction device and the implanted cardiac pacing lead through the tricuspid valve of the heart of the animal; and withdrawing the extraction device, accessory device, and the implanted cardiac pacing lead from the body vessel.

The following detailed description and the appended drawings describe and illustrate various example extraction devices useful for extracting implanted medical devices from a target location within a body lumen, such as that of a human being, and methods of extracting an implanted medical device from a target location within said body lumen. The description and illustration of these examples are provided to enable one skilled in the art to make and use example extraction devices and to practice example methods of extracting an implanted medical device from a target location within a body lumen. The inclusion of detailed descriptions and illustrations of these selected and specific examples is not intended to limit the scope of the invention, or its protection, in any manner. The invention is capable of being practiced or carried out in various ways and the examples described and illustrated herein are not considered exhaustive.

The components of the embodiments as generally described and illustrated in the figures herein can be arranged and designed in a wide variety of different configurations. Thus, the following more detailed description of various embodiments, as represented in the figures, is not intended to limit the scope of the present disclosure but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical and fluidic interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive). The phrase “fluid communication” is used in its ordinary sense and is broad enough to refer to arrangements in which a fluid (e.g., a gas or a liquid) can flow from one element to another element when the elements are in fluid communication with each other.

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

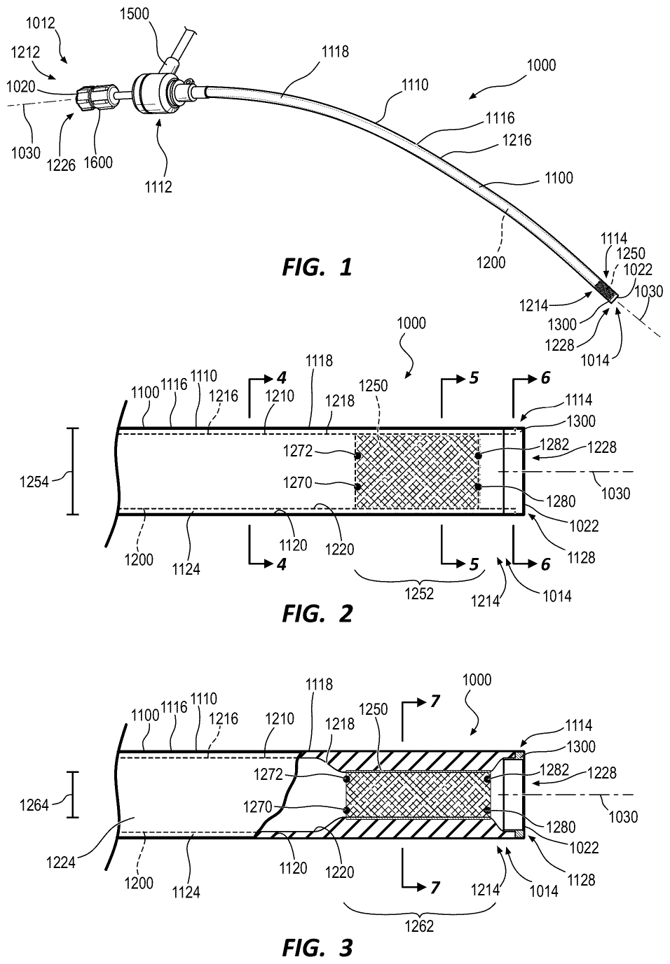

FIG. 1 illustrates a perspective view of an example extraction device 1000, according to embodiments described herein.

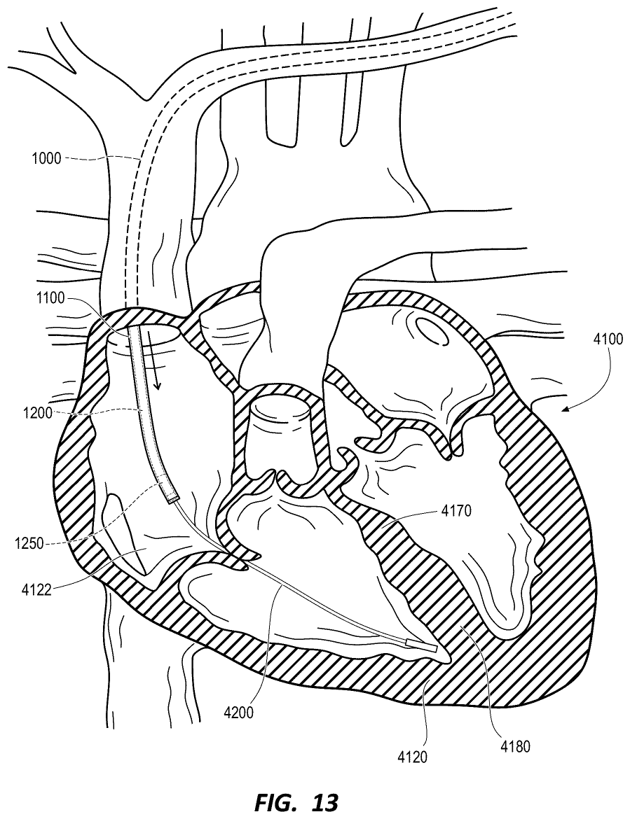

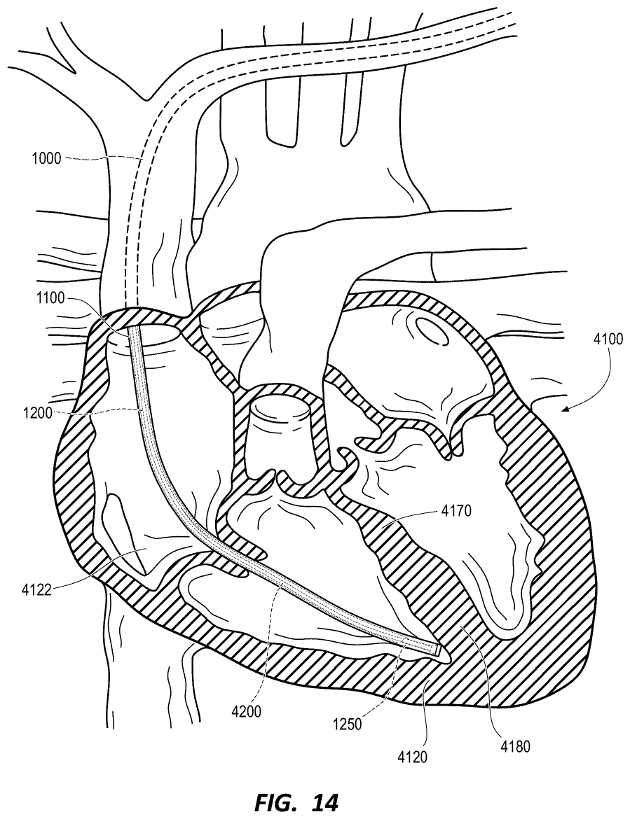

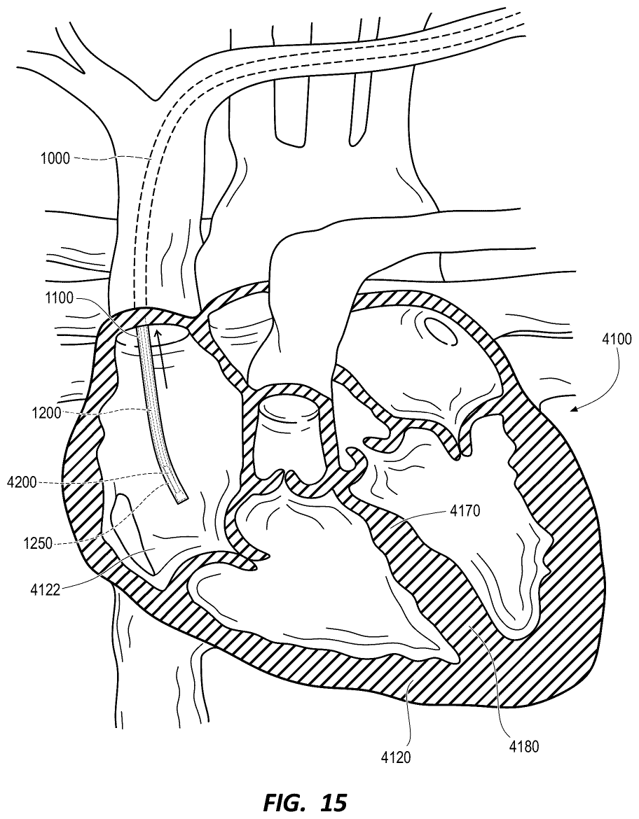

Similarly to FIG. 1, each of FIG. 2 and FIG. 3 illustrates a distal portion of an embodiment of the extraction device 1000 and shows a structural arrangement of the components of the extraction device 1000 during a stage of use of the extraction device 1000. Each of FIG. 13, FIG. 14, and FIG. 15 illustrates a portion of the extraction device 1000 disposed within a human heart 4100 during the performance of an example method of extracting an implanted medical device from a target location within a body lumen of a patient or an animal. In each of these figures, the extraction device 1000 is illustrated during a stage of performance of method 3000 to extract a pacing lead 4200 that is implanted in the right ventricle 4120 of heart 4100, in a post-AV node location on or near the Bundle of His 4170 (as will be discussed with respect to FIGS. 14-15.

The extraction device 1000 includes an outer sheath 1100 and an inner member 1200 disposed within, and axially movable relative to, the outer sheath 1100. The inner member 1200 includes an engagement portion 1250 that is movable between a first configuration and a second configuration. In the first configuration (illustrated in FIG. 2), the engagement portion 1250 is in a radially non-constricted arrangement in which the engagement portion 1250 extends longitudinally from the remainder of the inner member 1200 in a substantially continuous manner. In the second configuration (illustrated in FIG. 3), the engagement portion 1250 is in a radially constricted arrangement in which the engagement portion 1250 has an inner diameter that is less than the inner diameter of the engagement portion 1250 when in the first configuration. In the second configuration, the engagement portion 1250 can include a length extending along a longitudinal axis of the inner member 1200 that is longer than the length extending along the longitudinal axis of the inner member when in the first configuration.

In some cases, the engagement portion 1250 moves from the first configuration to the second configuration with retraction of the inner member 1200 relative to the outer sheath 1100, such as when a pulling force is applied to the inner member 1200, while maintaining a position of the outer sheath 1100. A distal support member 1300 is attached to both the outer sheath 1100 and the inner member 1200, thus enabling axial retraction of the inner member 1200 relative to the outer sheath 1100.

In the example extraction device 1000, an optional hub 1500 is disposed on a proximal end of the outer sheath 1100 and an optional handle 1600 is disposed on a proximal end of the inner member 1200. Inclusion of a hub having a hemostasis valve in an extraction device according to an embodiment is considered advantageous at least because these hubs can reduce or minimize fluid loss, such as blood loss, during a procedure in which the extraction device is used.

Extraction device 1000 has a proximal end 1012 and a distal end 1014. The terminal surface 1020 of the proximal end 1012 is formed by the handle 1600. The terminal surface 1022 of the distal end 1014 is formed by the distal support member 1300. The extraction device proximal end 1012 extends from the extraction device proximal terminal surface 1022 toward the extraction device distal end 1014. Similarly, the extraction device distal end 1014 extends from the extraction device distal terminal surface 1022 toward the extraction device proximal end 1012.

Extraction device 1000 has longitudinal axis 1030 that extends longitudinally through the geometric center of the cross-sectional area of the extraction device 1000 and along the length of the extraction device 1000. Longitudinal axis 1030 is also a longitudinal axis for each of the outer sheath 1100 and inner member 1200. A force can be applied to a component of the extraction device 1000 that is parallel to the longitudinal axis 1030 and proximally directed, or toward a user of extraction device 1000, while a force can also be applied to a component of the extraction device 1000 that is parallel to longitudinal axis 1030 and distally directed, or away from a user of extraction device 1000.

Outer sheath 1100 is a tubular member having a main body 1110 that extends from a proximal end 1112 to a distal end 1114 along longitudinal axis 1030. Main body 1110 defines a circumferential wall 1116 having an external surface 1118, an internal surface 1120, and a thickness 1122 extending between the external surface 1118 and internal surface 1120. Circumferential wall 1116 defines a lumen 1124 that is bounded by internal surface 1120. The lumen 1124 extends from the proximal end 1112 of the outer sheath 1100 to the distal end 1114 of the outer sheath 1100. Proximal end 1112 defines proximal opening (not visible in drawings) that provides access to lumen 1124 and distal end 1114 defines distal opening 1128 that provides access to lumen 1124. Lumen 1124 is sized and configured to receive inner member 1200 and to allow inner member 1200 to be retracted within lumen 1124 to move engagement portion 1250 from its first configuration to its second configuration, as described herein.

Extraction device 1000 is sized and configured for use in percutaneous procedures. As such, outer sheath 1100 is sized and configured to be inserted into and navigated within a body vessel of an animal, such as a blood vessel. For example, extraction device 1000 is particularly well suited for extraction of cardiac pacing leads. Accordingly, outer sheath 1100 is sized and configured to be inserted into and navigated within a blood vessel, to allow the distal end 1014 of the extraction device to be inserted into a chamber of the heart and passed over a pacing lead, or portion of a pacing lead, to be extracted from the heart.

Inner member 1200 is a tubular member having a main body 1210 that extends from a proximal end 1212 to a distal end 1214 along longitudinal axis 1030. Main body 1210 defines a circumferential wall 1216 having an external surface 1218, an internal surface 1220, and a thickness 1222 extending between the external surface 1218 and internal surface 1220. Circumferential wall 1216 defines a lumen 1224 that is bounded by internal surface 1220. The lumen 1224 extends from the proximal end 1212 of the inner member 1200 to the distal end 1214 of the inner member 1200. Proximal end 1212 defines proximal opening 1226 that provides access to lumen 1224 and distal end 1114 defines distal opening 1228 that provides access to lumen 1224. Lumen 1224 is sized and configured to receive an implanted medical device, or portion of an implanted medical device, for which the extraction device 1000 is intended to be used, such as a portion of a cardiac pacing lead, as described herein.

A distal portion of inner member 1200 defines engagement portion 1250. Engagement portion 1250 is structurally distinct from the remainder of the inner member 1200 and is adapted to be constricted around a member disposed within lumen 1224 of inner member 1200. As illustrated in FIG. 2, engagement portion 1250 has a first configuration in which the engagement portion 1250 has a first axial length 1252 and a first inner diameter 1254. As illustrated in FIG. 3, engagement portion 1250 has a second axial length 1262 and a second inner diameter 1254. The second axial length 1262 is greater than the first axial length 1252 and the second inner diameter 1264 is less than the first inner diameter 1254. The engagement portion 1250 moves from the first configuration to the second configuration with retraction of the inner member 1200 relative to the outer sheath 1100, such as by application of a pulling force on the inner member 1200 while maintaining a position of the outer sheath 1100.

Engagement portion 1250 can have any suitable structure that provides the desired transition between the first and second configurations, as described herein, and a skilled artisan will be able to select a suitable structure for an engagement portion in an extraction device according to a particular embodiment based on various considerations, including any desired structural interface with an implanted medical device with which the extraction device is intended to be used. During use, the engagement portion contacts the external surface of an implanted medical device being extracted with the extraction device. Accordingly, any structure of the engagement portion that increases surface area of the contact interface between the engagement portion and the implanted medical device may be advantageous.

As illustrated in FIG. 2, FIG. 3, FIG. 5A, and FIG. 7, engagement portion 1250 (in example extraction device 1000) is a mesh of one or more wire members. Each wire member has a round cross-sectional shape, such as a circular cross-sectional shape. This structure is considered advantageous at least because portions of the wire member or wire members readily slide over each other during transition from the first configuration to the second configuration, facilitating use of the extraction device.

Other structures are also considered suitable. For example, FIG. 5B illustrates a sectional view of an exemplary embodiment of an extraction device. More specifically, FIG. 5B illustrates an alternative engagement portion 1250′ that is a mesh of one or more wire members having a rectangular cross-sectional shape, as opposed to a circular cross-sectional shape. This structure is considered advantageous at least because the rectangular cross-sectional shape of the one or more wire members provides a flat surface on the inner side of each wire member, which provides relatively more surface area for engagement with an implanted medical device during use of the extraction device as compared to an engagement portion comprising wire members having a round cross-sectional shape.

While this additional surface area is considered advantageous, it is noted that it can also be advantageous to include one or more wire members having a rectangular cross-sectional shape, or other polygonal cross-sectional shape, arranged such that an angle between sides is directed inward toward the longitudinal axis 1030 of the extraction device. This structural arrangement of one or more wire members in an engagement portion can be advantageous at least because the positioning of an angle between sides of the cross-sectional shape of the one or more wire members can aid in gripping of an implanted medical device to be extracted by the engagement portion, such as by biting of the angle into an outer surface of the implanted medical device.

Returning to the descriptions of FIG. 2, FIG. 3, FIG. 5A, and FIG. 7, engagement portion 1250 can include surface modifications or additional elements that enhance contact between the engagement portion 1250 and an implanted medical device being extracted by extraction device 1000. For example, wire members of engagement portion 1250 can have a roughened outer surface. Also, an adhesive can be applied to the outer surface or portions of the outer surface of the wire members of engagement portion 1250. One or more markers 1270 can be associated with the inner member 1200. For example, as illustrated in FIG. 2 and FIG. 3, a plurality of markers 1270, 1272 can be disposed on the proximal end of the engagement portion 1250. Also in this embodiment, a plurality of markers 1280, 1282 can be disposed on the distal end of the engagement portion 1250. In some embodiments, some or all of any included markers are disposed on, in, or otherwise associated with another portion of the inner member 1200 that is not the engagement portion. For example, one or more markers can be disposed on, in, or otherwise associated with the circumferential wall of the inner member in a position that is generally adjacent the proximal end of the engagement portion 1250. Also, if included, the marker or markers can have any suitable size, shape, and configuration. For example, radiopaque markers can be used. Any suitable number of markers can be included. Also, while the illustrated markers 1270, 1272, 1280, 1282 comprise plug or dot markers, it is noted that rings or others suitable shapes can be used.

Distal support member 1300 is attached to the distal end 1114 of the outer sheath 1100 and the distal end 1214 of the inner member 1200. As illustrated in FIG. 6 and FIG. 8, distal support member 1300 is a ring member that defines a circumferential wall 1316 having an external surface 1318, an internal surface 1320, and a lumen 1324 that is bounded by internal surface 1320. The lumen 1324 extends between a proximal opening 1326 that provides access to lumen 1324 a distal opening 1328 that provides access to lumen 1324. The lumen 1324 of distal support member is continuous with the lumen 1124 of the outer sheath 1100 and the lumen 1224 of the inner member 1200.

As illustrated in FIG. 8, distal support member 1300 defines circumferential shoulder 1360 that receives the distal end 1114 of outer sheath 1100 such that outer surface 1118 of outer sheath 1100 is continuous with outer surface 1318 of distal support member 1300. Distal end 1114 of outer sheath 1100 is secured to the proximal end 1312 of distal support member 1300. The distal end 1114 of outer sheath 1100 can be secured to the distal support member 1300 in any suitable manner, such as with an adhesive, welding, bonding, and the like.

Also as illustrated in FIG. 8, distal end 1214 of inner member 1200 is secured to proximal end 1312 of distal support member 1300, which maintains the distal end 1214 of inner member 1200 in a fixed position relative to distal support member 1300 and outer sheath 1100 as engagement portion 1250 moves from its first configuration, illustrated in FIG. 2, to its second configuration, illustrated in FIG. 3. The distal end 1214 of inner member 1200 can be secured to the distal support member 1300 in any suitable manner. In the illustrated example, ends of wire members of engagement portion are adhered to inner surface 1320 of distal support member 1300. Other examples of suitable attachments include, but are not limited to, mechanical attachments, such as attachments formed by crimping, embedding, and the like, bonding, welding, swaging, and other attachments.

In some embodiments, distal end 1314 of distal support member defines a cutting surface that facilitates separation of tissue from an implanted medical device that is being extracted from a target location in a body lumen by the extraction device 1000.

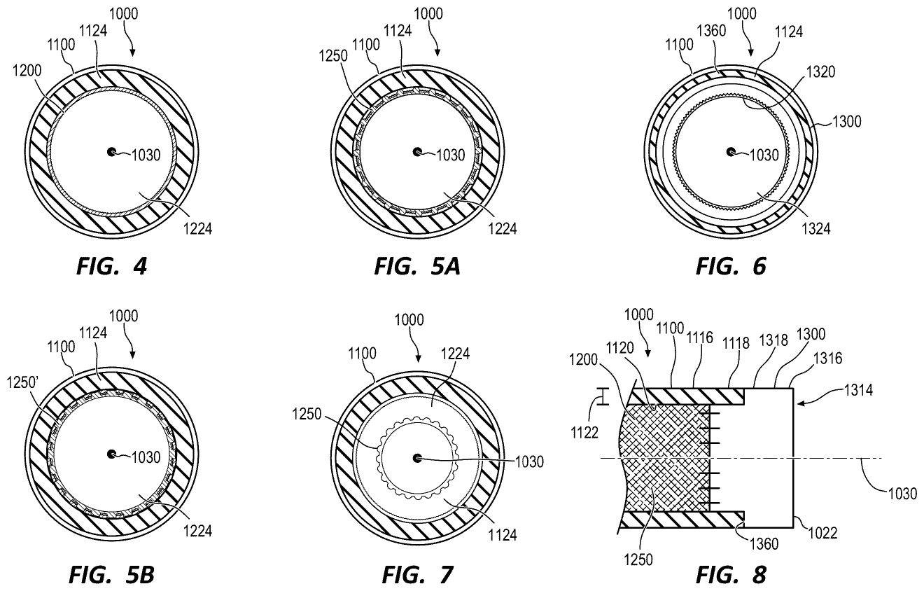

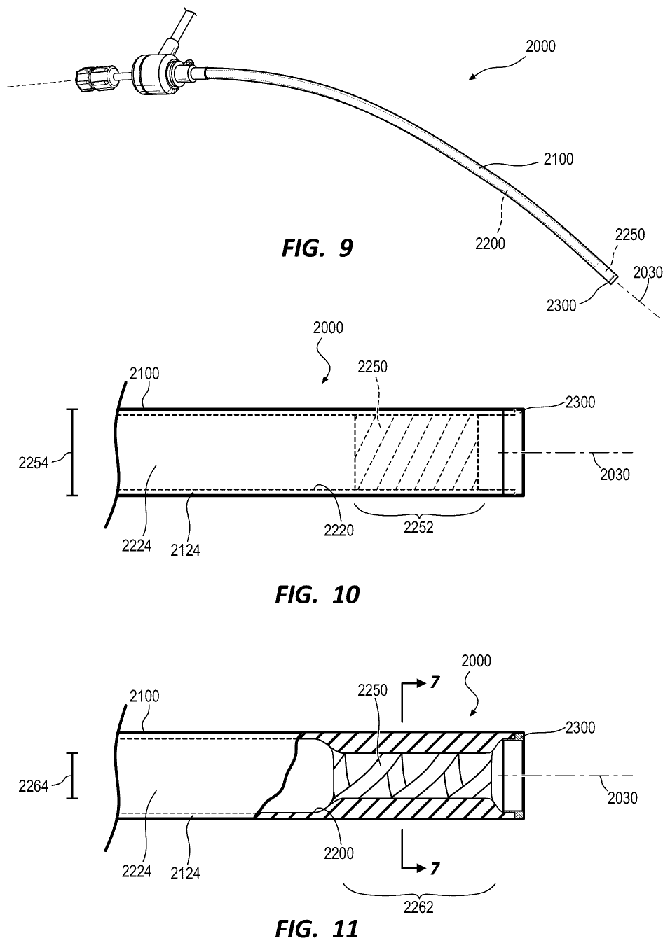

FIGS. 9 through 11 (as well as FIG. 5B) illustrate embodiments of an extraction device 2000 that resembles extraction device 1000 described above, in certain respects. Accordingly, like features are designated with like reference numerals, with the leading digits incremented to “1” through “2”. For example, the embodiment depicted in FIG. 9 includes engagement portion 2250 that may, in some respects, resemble engagement portion 1250 of FIGS. 1-8. Relevant disclosure set forth above regarding similarly identified features thus may not be repeated hereafter. Moreover, specific features of extraction device 1000 and related components shown in FIGS. 1-8 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the distal tips and related components depicted in FIGS. 9-11.

Any suitable combination of the features, and variations of the same, described with respect to extraction device 1000 and related components illustrated in FIGS. 1-8 can be employed with the extraction device and related components of FIGS. 9-11, and vice versa. This pattern of disclosure applies equally to further embodiments depicted in subsequent figures and described hereafter, wherein the leading digits may be further incremented.

FIGS. 9-11 illustrate an embodiment of an extraction device 2000 and will be described in tandem. Accordingly, FIG. 9 illustrates a perspective view of an exemplary embodiment of an extraction device, according to embodiments described herein. FIG. 10 illustrates a lateral view of a distal end portion of the example extraction device of FIG. 9 with the engaging portion in a first configuration, according to embodiments described herein. FIG. 11 illustrates a lateral view of a distal end portion of the example extraction device of FIG. 9 with the engaging portion in a second configuration, according to embodiments described herein.

Extraction device 2000 includes an outer sheath 2100 and an inner member 2200 disposed within inner lumen 2121 of outer sheath 2100 and axially movable relative to the outer sheath 2100. The inner member 2200 includes an engagement portion 2250 that is movable between a first configuration and a second configuration. In the first configuration, illustrated in FIG. 10, the engagement portion 2250 is in a radially non-constricted arrangement in which the engagement portion 2250 extends longitudinally from the remainder of the inner member 2200 in a substantially continuous manner. In the second configuration, illustrated in FIG. 11, the engagement portion 2250 is in a radially constricted arrangement in which the engagement portion 2250 has an inner diameter that is less than the inner diameter of the engagement portion 2250 when in the first configuration and a length extending along a longitudinal axis of the inner member 2200 that is longer than the length extending along the longitudinal axis of the inner member when in the first configuration. The engagement portion 2250 moves from the first configuration to the second configuration with retraction of the inner member 2200 relative to the outer sheath 2100, such as by application of a pulling force on the inner member 2200 while maintaining a position of the outer sheath 2100. A distal support member 2300 is attached to both the outer sheath 2100 and the inner member 2200, enabling axial retraction of the inner member 2200 relative to the outer sheath 2100.

Engagement portion 2250 is structurally distinct from the remainder of the inner member 2200 and is adapted to be constricted around an implanted medical device disposed within lumen 2224 of inner member 2200. As illustrated in FIG. 10, engagement portion 2250 has a first configuration in which the engagement portion 2250 has a first axial length 2252 and a first inner diameter 2254. As illustrated in FIG. 11, engagement portion 2250 has a second axial length 2262 and a second inner diameter 2264. The second axial length 2262 is greater than the first axial length 2252 and the second inner diameter 2264 is less than the first inner diameter 2254. The engagement portion 2250 moves from the first configuration to the second configuration with retraction of the inner member 2200 relative to the outer sheath 2100, such as by application of a pulling force on the inner member 2200 while maintaining a position of the outer sheath 2100.

In this example, engagement portion 2250 comprises a coil defined by a ribbon of material having a flat inner surface and wrapping in a helical pattern around a longitudinal axis 2030 of the extraction device 2000. The longitudinal distance between adjacent turns of the coil is greater when the engagement portion 2250 is in the second configuration.

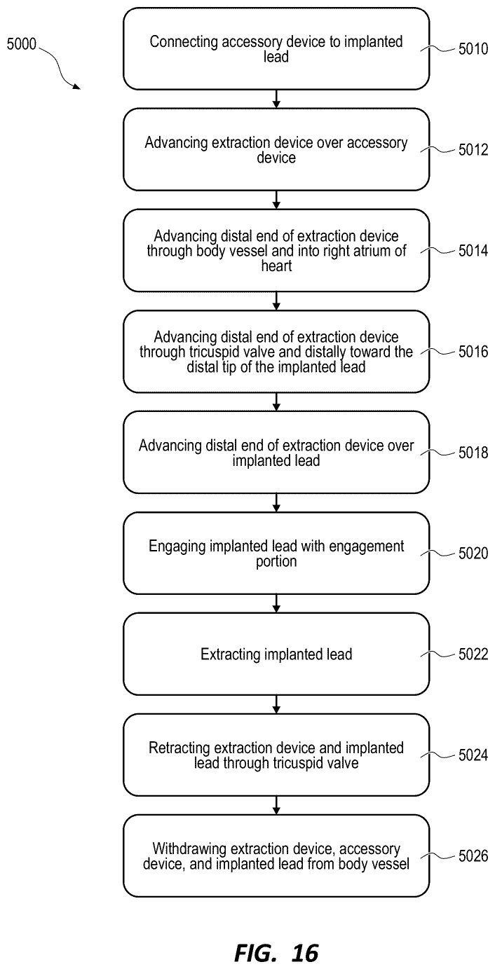

Extraction devices according to embodiments herein can be a component in an extraction system that includes a separate surrounding sheath defining a sheath lumen. In these extraction systems, an extraction device according to an embodiment is disposed within the sheath lumen defined by the surrounding sheath. In use during an extraction procedure, such as the example methods described herein, the surrounding sheath is used to dilate surrounding vessel wall tissue as the extraction device is advanced through a body vessel, such as a vascular vessel of the venous system. Inclusion of a surrounding sheath in these extraction systems is particularly advantageous for use of an extraction device in methods of extracting an implanted cardiac pacing lead, such as the example methods described herein. Inclusion of a surrounding sheath in these extraction systems is particularly advantageous for use of an extraction device in methods of extracting a cardiac pacing lead implanted in a post-AV node location of the heart of an animal, such as example method 5000 illustrated in FIG. 16 and described below.

In addition to enabling dilation of surrounding vessel wall tissue during advancement of an engagement device through a body vessel, the surrounding sheath can be used to provide counter traction during detachment of an implanted medical device from tissue, which can be particularly important during extraction of cardiac pacing leads, for example, and more particularly important during extraction of cardiac pacing leads implanted in a post-AV node location within the heart. For these methods, the surrounding sheath can be used to contact and support or stabilize the heart wall by enabling the application of a distally-directed force on the surrounding sheath while the distal tip of the lead is being detached from any lead-to-heart wall adhesions that may have formed while the target lead was indwelling by application of a proximally-directed force on the inner member of the extraction device. Use of the surrounding sheath to provide counter traction in this manner may aid in the prevention of a cardiac avulsion, for example, in which the distal tip of an extracted lead carries a section of heart tissue with it.

The components of an extraction device according to an embodiment may be manufactured from a variety of different materials, including conventional materials for minimally-invasive interventional medical devices such as catheters, sheaths, and guidewires. Examples of types of materials considered suitable for use in the components of extraction devices according to embodiments include, but are not limited to, polymeric materials and metals. Examples of suitable polymeric materials include, but are not limited to, polyurethane, silicone rubber, polytetrafluoroethylene, polyether block amide, polyvinyl chloride, polyethylene, nylon, ethylene vinyl acetate, polycarbonate, and thermoplastic elastomers. Examples of suitable metals include, but are not limited to, stainless steel, nitinol, titanium, and cobalt-chromium alloys. A skilled artisan will be able to select a suitable material for each component of an extraction device according to a particular embodiment based on various considerations, including any desired flexibility, torqueability, and visibility under or in conjunction with various medical imaging modalities.

In some embodiments, the outer sheath and inner member of an extraction device according to an embodiment are advantageously made of a polymeric material. The engagement portion of an extraction device according to an embodiment is advantageously made of metal, such as a shape memory alloy. Nickel-titanium alloys, such as nitinol, are considered particularly advantageous for use in the engagement portion. The distal support member is advantageously made of a polymeric material with metal markers disposed therein. Alternatively, the distal support member can be entirely formed of a metal, such as a metal having desirable imaging characteristics for an imaging modality of interest.

An extraction device according to an embodiment can have any suitable length. A skilled artisan can select a suitable length for an extraction device according to a particular embodiment based on various considerations, such as the location with a body of an implanted medical device intended to be extracted using the extraction device, a planned approach for an extraction procedure in which the extraction device is intended to be used, or any expected dimensions of a body vessel within which the extraction device is intended to be used. For example, for an extraction device according to an embodiment and intended for use in a method of extracting an implanted medical device, such as a cardiac pacing lead, from the body of a human being using a superior approach, a length of greater than about 35 cm is suitable at least because this length provides sufficient length for a user to manipulate the extraction device to extract a pacing lead through a superior approach. A length of between about 35 cm and about 45 cm is considered particularly advantageous for an extraction device according to an embodiment and intended for use in a method of extracting an implanted cardiac pacing lead from the body of a human being using a superior approach.

In some embodiments, for an extraction device according to an embodiment and intended for use in a method of extracting an implanted medical device, such as a cardiac pacing lead, from the body of a human being using an inferior approach, such as a femoral approach, a length of greater than about 90 cm is suitable at least because this length provides sufficient length for a user to manipulate the extraction device to extract a pacing lead through a femoral approach. Other lengths considered suitable for extraction devices according to an embodiment and intended for use in a method of extracting an implanted cardiac pacing lead from the body of a human being using an inferior approach include, but are not limited to, a length of between about 90 cm and about 150 cm, a length of between about 90 cm and about 120 cm, a length of between about 90 cm and about 100 cm, a length of between about 90 cm and about 95 cm; and a length of between about 90 cm and about 92 cm.

Methods of extracting an implanted medical device from a target location within a body lumen of an animal are also provided.

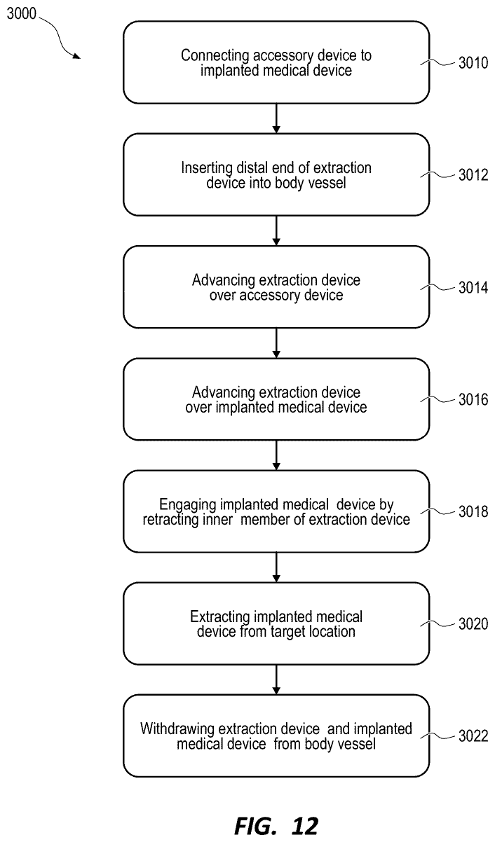

FIG. 12 illustrates an example method for extracting an implanted medical device, according to embodiments described herein. FIG. 12 is a flowchart illustration of an example method 3000 of extracting an implanted medical device from a target location within a body lumen of an animal. An initial step 3010 comprises connecting an accessory device, such as a locking stylet or an extended, to the implanted medical device. Another step 3012 comprises inserting the distal end of an extraction device according to an embodiment into a body vessel of an animal, such as a human being. Another optional step 3014 is included if optional step 3010 is included and comprises advancing the distal end of the extraction device through the body vessel and over the accessory device. Another step 3016 comprises advancing the distal end of the extraction device through the body vessel and over the implanted medical device. Another step 3018 comprises engaging the implanted medical device with the engagement portion of the extraction device by retracting the inner member of the extraction device relative to the outer sheath of the extraction device, such as by application of a pulling force on the inner member of the extraction device while maintaining a position of the outer sheath of the extraction device. Another step 3020 comprises extracting the implanted medical device from tissue within which the medical device is implanted by retracting the extraction device within the body vessel. Another step 3022 comprises withdrawing the extraction device and the implanted medical device from the body vessel. If optional step 3010 is included, this step 3022 includes withdrawing the accessory device.

Inclusion of optional step 3010 is considered advantageous when the proximal end of the implanted medical device to be extracted is located within the body vessel, for example. In this situation, connecting an accessory device to the implanted medical device allows for the creation of a rail-like structure comprising the implanted medical device and the accessory device having a proximal end located outside of the body vessel, which facilitates the step 3016 of advancing the extraction device over the implanted medical device.

If step 3010 is included, the accessory device preferably comprises an elongate member having a distal end or other axial portion that is adapted to be connected to a proximal end or other axial portion of the implanted medical device. For example, a locking stylet configured to be passed into a lumen defined by an implanted medical device and connected to the implanted medical device can be used as an accessory device in the method 3000. The Liberator locking stylet (Cook Medical, Bloomington, Indiana) is one example of a locking stylet considered suitable for use as an accessory device in the inventive methods. Use of a locking stylet as an accessory device is considered particularly advantageous when the implanted medical device to be extracted defines a lumen, such as conductor coil cardiac pacing leads.

Extender devices configured to be connected to an implanted medical device can also be used as an accessory device in the method 3000. The Bulldog extender (Cook Medical, Bloomington, Indiana) is one example of an extender device considered suitable for use as an accessory device in the inventive methods. Use of an extender device as an accessory device is considered particularly advantageous when the implanted medical device to be extracted does not define a lumen, such as lumenless cardiac pacing leads. Use of an extender device as an accessory device is also considered particularly advantageous when the implanted medical device is a broken cardiac pacing lead.

FIGS. 13, 14, and 15 illustrate a portion of example extraction device 1000 disposed within a human heart 4100 during extraction of an implanted medical device from a target location within a body lumen, and will be described in tandem. Accordingly, FIG. 13 illustrates a schematic illustration of an extraction device disposed within a human heart and in a first position relative to implanted leads, according to embodiments described herein. FIG. 14 illustrates a schematic illustration of an extraction device disposed within a human heart and in a second position relative to implanted leads, according to embodiments described herein. FIG. 15 illustrates a schematic illustration of an extraction device disposed within a human heart as the pacing leads are being extracted relative to the heart, according to embodiments described herein.

In each of these figures, the extraction device 1000 is illustrated during a stage of performance of method 3000 to extract a pacing lead 4200 that is implanted in the right ventricle 4120 of heart 4100, distal to the right atrium 4122 in a post-AV node location beyond the Bundle of His 4170 and on or near the right bundle branch 4180. This is an example of a placement of a pacing lead used in CSP therapy modalities, and the extraction devices and methods described herein are particularly well-suited for use in the extraction of cardiac pacing leads implanted in this and other post AV node locations used in these CSP modalities. FIG. 14 illustrates the extraction device 1000 being advanced to the target location and over the implanted pacing lead 4200, such as in step 3012 of method 3000. FIG. 15 illustrates the inner member of the extraction device 1000 being retracted relative to the outer sheath of the extraction device, such as by application of a pulling force on the inner member of the extraction device while maintaining a position of the outer sheath of the extraction device, to engage the pacing lead 4200 with the engagement portion of the extraction device 1000. The distal support member 1300 of the extraction device is contacting the wall of the heart 4100 in the base of the right ventricle 4120. This is reflective of step 3014 of method 3000.

FIG. 16 illustrates an example method for extracting an implanted medical device, according to embodiments described herein. More specifically, in some cases, the method 5000 of FIG. 16 can correspond to an implanted medical device being withdrawn from the heart 4100, such as in step 3018 of method 3000.

Example methods using extraction devices according to embodiments are particularly well suited for extraction of cardiac pacing leads implanted in the tissue of the heart of an animal, such as a human being. Example methods are particularly well suited for extraction of cardiac pacing leads implanted under a CSP therapy modality, such as cardiac pacing leads implanted on or near the Bundle of His, the right bundle branch, the left bundle branch, or another post-AV node conduction pathway position.

As shown in FIG. 16, an example method 5000 can be used to extract a cardiac pacing lead implanted in a post AV node location of the heart. An initial step 5010 comprises connecting an accessory device to the implanted cardiac pacing lead. Another step 5012 comprises advancing an extraction device according to an embodiment over the accessory device. Another step 5014 comprises advancing the distal end of the extraction device through the body vessel and into the right atrium of the heart of the animal. Another step 5016 comprises advancing the distal end of the extraction device through the tricuspid valve and distally toward the distal end of the implanted cardiac pacing lead. Another step 5018 comprises advancing the distal end of the extraction device over the implanted cardiac pacing lead. Another step 5020 comprises engaging the implanted cardiac pacing lead with the engagement portion of the extraction device by retracting the inner member of the extraction device relative to the outer sheath of the extraction device, such as by application of a pulling force on the inner member of the extraction device while maintaining a position of the outer sheath of the extraction device. Another step 5022 comprises extracting the implanted cardiac pacing lead from an implantation location in tissue defining the right ventricle of the heart of the animal by retracting the extraction device within the body vessel. Another step 5024 comprises retracting the extraction device and the implanted cardiac pacing lead through the tricuspid valve of the heart of the animal. Another step 5026 comprises withdrawing the extraction device and the implanted cardiac pacing lead from the body vessel.

Similar to optional step 3010 in method 3000 illustrated in FIG. 12 and described above, the accessory device in step 5010 advantageously comprises an elongate member having a distal end or other axial portion that is adapted to be connected to a proximal end or other axial portion of the implanted cardiac pacing lead. As described above, use of a locking stylet as an accessory device is considered particularly advantageous when the implanted cardiac pacing lead to be extracted defines a lumen, such as conductor coil cardiac pacing leads. Also as described above, use of an extender device as an accessory device is considered particularly advantageous when the implanted cardiac pacing lead to be extracted does not define a lumen, such as lumenless cardiac pacing leads. Use of an extender device as an accessory device is also considered particularly advantageous when the implanted cardiac pacing lead is a broken cardiac pacing lead.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular arrangements of elements and steps disclosed herein have been selected by the inventor simply to describe and illustrate examples of the invention and are not intended to limit the scope of the invention or its protection, which is to be given the full breadth of the appended claims and any and all equivalents thereof.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

What is claimed is:

1. An extraction device comprising:
an outer sheath including:
an outer sheath proximal end,
an outer sheath distal end, and
an outer sheath body defining an outer sheath lumen;
an inner tubular member disposed within the outer sheath lumen, the inner tubular member having:
an inner tubular member proximal end,
an inner tubular member distal end, and
an inner tubular member body defining an inner tubular member lumen;
an engagement member defined by the inner tubular member and disposed adjacent to the inner tubular member distal end, the engagement member defining an engagement member lumen and being configured to move between a first configuration in which the engagement member lumen has a first diameter and a first length and a second configuration in which the engagement member lumen has a second diameter and a second length, in response to a proximally directed force upon the inner tubular member proximal end; and
a support member defining a support member exterior perimeter attached to the outer sheath distal end, and a support member interior perimeter attached to the inner tubular member distal end.

2. The extraction device of claim 1, further comprising a handle attached to the outer sheath proximal end and the inner tubular member proximal end, configured to apply a proximally directed force upon the inner tubular member proximal end in relation to the outer tubular member proximal end such that the engagement member moves from the first configuration to the second configuration.

3. The extraction device of claim 2, wherein the handle is configured to apply a distally directed force upon the inner tubular member proximal end in relation to the outer tubular member proximal end such that the engagement member moves from the second configuration to the first configuration.

4. The extraction device of claim 1, wherein the engagement member is a mesh.

5. The extraction device of claim 1, wherein the engagement member is a weave.

6. The extraction device of claim 1, wherein the support member is radiopaque.

7. The extraction device of claim 1, further comprising a marker disposed proximally adjacent to the engagement member.

8. An extraction device comprising:
an outer sheath including:
an outer sheath proximal end,
an outer sheath distal end, and
an outer sheath body defining an outer sheath lumen;
an inner tubular member disposed within the outer sheath lumen, the inner tubular member having:
an inner tubular member proximal end,
an inner tubular member distal end, and
an inner tubular member body defining an inner tubular member lumen;
an engagement member defined by the inner tubular member and disposed adjacent to the inner tubular member distal end, the engagement member defining an engagement member lumen and being configured to move between a first configuration in which the engagement member lumen has a first diameter and a first length and a second configuration in which the engagement member lumen has a second diameter and a second length, in response to a proximally directed force upon the inner tubular member proximal end;
a support member defining a support member exterior perimeter attached to the outer sheath distal end, and a support member interior perimeter attached to the inner tubular member distal end; and
a guide member disposed within the inner tubular member lumen, the guide member defining a guide member distal terminal surface.

9. The extraction device of claim 8, wherein the guide member terminal surface forms a blade configured to cut through tissue.

10. The extraction device of claim 8, further comprising a handle attached to the outer sheath proximal end and the inner tubular member proximal end, configured to apply a proximally directed force upon the inner tubular member proximal end in relation to the outer tubular member proximal end such that the engagement member moves from the first configuration to the second configuration.

11. The extraction device of claim 9, wherein the handle is configured to apply a distally directed force upon the inner tubular member proximal end in relation to the outer tubular member proximal end such that the engagement member moves from the second configuration to the first configuration.

12. The extraction device of claim 8, wherein the engagement member is a mesh.

13. The extraction device of claim 8, wherein the engagement member is a weave.

14. The extraction device of claim 8, wherein the support member is radiopaque.

15. The extraction device of claim 8, further comprising a first marker disposed on the inner tubular member proximally adjacent to the engagement member.

16. The extraction device of claim 8, further comprising a second marker and a third marker disposed on the inner tubular member, the second marker being rotatably disposed generally 90 degrees from the first marker, and the third marker being rotatably disposed generally opposite the second marker.

17. A method of extracting an implanted medical device comprising:
inserting an extraction device into a vascular system, the extraction device including:
an outer sheath including an outer sheath proximal end, an outer sheath distal end, and an outer sheath body defining an outer sheath lumen;
an inner tubular member disposed within the outer sheath lumen, the inner tubular member having an inner tubular member proximal end, an inner tubular member distal end, and an inner tubular member body defining an inner tubular member lumen;
an engagement member defined by the inner tubular member and disposed adjacent to the inner tubular member distal end, the engagement member defining an engagement member lumen and being configured to move between a first configuration in which the engagement member lumen has a first diameter and a first length and a second configuration in which the engagement member lumen has a second diameter and a second length, in response to a proximally directed force upon the inner tubular member proximal end; and
a support member defining a support member exterior perimeter attached to the outer sheath distal end, and a support member interior perimeter attached to the inner tubular member distal end;
positioning the extracting device over the implanted medical device such that the implanted medical device is partially concentrically encased within the engagement member;
applying a proximally directed force upon the inner tubular member in relation to the outer sheath such that the engagement member moves from the first configuration to the second configuration; and
applying a proximally directed force upon the extraction device.

18. The method of claim 17, further comprising extracting the extraction device from the animal body.

19. The method of claim 17, further comprising locating the extraction device within the body via a radiopaque marker.

20. The method of claim 17, further comprising advancing the extraction device about a guide member disposed within the inner tubular member lumen.
